(19) United States
(12) Patent Application Publication
(10) Pub. No.: US 2006/0018862 A1
(43) Pub. Date: Jan. 26, 2006

(54) Substrate based skin care device

(76) Inventors: Minghua Chen, Kobe (JP); Peiwen Sun, Chao Yang District (CN); Akira Date, Ashiya (JP); Etsuko Yuyama, Kobe (JP); Donald Lynn Bissett, Hamilton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/187,451

(22) Filed: Jul. 22, 2005

Related U.S. Application Data
(60) Provisional application No. 60/590,563, filed on Jul. 23, 2004.

(51) Int. Cl. A61K 8/60; A61K 31/7048

(52) U.S. Cl. 424/70.14; 514/27

(57) ABSTRACT

Disclosed is a skin care device comprising: (1) a dermatologically acceptable preformed substrate and (2) a flavonoid compound, wherein the device comprises a liquid medium for delivering the flavonoid compound.

### CROSS REFERENCE TO RELATED APPLICATION

This application claims the benefit of U.S. Provisional Application No. 60/590,563, filed on Jul. 23, 2004.

### FIELD OF THE INVENTION

The present invention relates to a substrate based skin care device that provides various improved skin care benefits such as skin lightening and anti-aging.

### BACKGROUND

Various treatment for the skin are proposed for delaying, minimizing or even eliminating skin hyperpigmentation (age spots, freckles, blotches, darkening, sallowness, uneven tone, and the like), wrinkling and other chronical changes typically associated with skin aging or environmental damage to human skin. Such treatments range from application of specialty cosmetics such as packs and masks, oral intake of vitamins, to chemical peeling, laser surgery, photofacial, and others. Generally, it is believed that effective treatment requires more time, physical, and financial commitment. There is a high desire for a treatment which is effective, but is safe and reasonably priced such that the consumer can use daily.

Various skin lightening agents and anti-aging agents are known in the art. It is also known that combination of actives may provide synergistic benefit. Flavonoids such as hesperidin are known in the art for use on the skin, for example in Japanese patent publications A11-346792, A2002-255827, A2003-137734, and United States Patent Application Publication 2002/13481.

Substrate based skin care devices, such as masks and patches, are increasing popularity as a slightly elaborate, but effective means of providing special treatment at the consumer's home. Generally, the consumer expects relatively high efficacy from these products. Substrate based skin care devices typically contain more than a saturated amount of aqueous liquid composition. When such substrate based skin care device is applied to a certain area of the skin, the temperature of the certain skin surface significantly drops because of such abundant water. While such temperature drop may provide a positive cooling sensation to the consumer, it may also act negatively from the standpoint of penetration of skin active agents. Namely, penetration of active agents through the skin is decelerated as temperature of the skin surface is decreased. From the standpoint of skin penetration via a substrate based skin care device, a skin active agent that alleviates temperature drop of the skin surface is desired.

Based on the foregoing, there is a need for a substrate based skin care device which provides improved skin conditioning benefits, while also alleviating temperature drop of the skin surface when applied to the skin. Specifically, there is a need for a substrate based skin care device which provides skin lightening benefit and/or anti-aging benefit.

None of the existing art provides all of the advantages and benefits of the present invention.

### SUMMARY

The present invention is directed to a skin care device comprising:
(1) a dermatologically acceptable preformed substrate; and
(2) from about 0.001% to about 10% of a flavonoid compound;
   wherein the device comprises a liquid medium for delivering the flavonoid compound.

The present invention is also directed to a method of providing skin lightening benefit comprising the steps of: applying to the skin the aforementioned device.

The present invention is also directed to a method of providing anti-aging benefit to the skin comprising the steps of: applying to the skin the aforementioned device.

These and other features, aspects, and advantages of the present invention will become evident to those skilled in the art from a reading of the present disclosure with the appended claims.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

The term “preformed” as used herein, means that an article is manufactured into a form having a predetermined shape and size, wherein the article may be removed from any associated packaging and placed or draped onto the target surface by the fingers without further preparative steps by the user. The term “preformed” also means that, when manufacturing is completed, the article substantially retains its shape at the desired normal storage temperature when lying on a flat surface. The article may nevertheless flex or be deformed when applied to an uneven surface or if impressed.

Flavonoid Compound

The present device comprises a flavonoid compound. The flavonoid compound is used at a level of from about 0.001% to about 10%, preferably from about 0.01% to about 5%, more preferably from about 0.05% to about 1%, by weight of the liquid composition, gel sheet, or coating composition of the present device, as described hereinafter. Flavonoid compounds are known to provide antioxidant, UV absorbing, and radical scavenging benefits. Flavonoid compounds are also known to be effective in strengthening collagen structure.

Flavonoid compounds useful herein are derived from either 2-phenylbenzopyrone (I) or 3-phenylbenzopyrone (II) skeleton structure as follows. (McGraw-Hill encyclopedia of Science and technology)

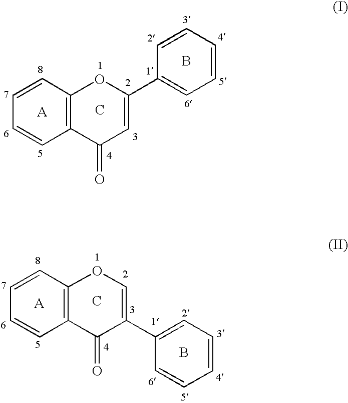

Flavonoid compounds can be further classified into different groups, depending on the oxidation level or substitution pattern of their heterocylic ring (ring C). Flavonoid compounds useful herein include unsubstituted flavanones, substituted flavanones, unsubstituted flavones, substituted flavones, unsubstituted chalcones, substituted chalcones, unsubstituted isoflavones, and substituted isoflavones. By the term “substituted” as used herein means flavonoid compounds wherein one or more hydrogen atoms of the skeleton structure as described above has been independently replaced with hydroxyl, C1-C8 alkyl, C1-C4 alkoxyl, O-glycoside, and the like or a mixture of these substituents. Flavonoid compounds particularly useful herein are selected from the group consisting of substituted flavanones, substituted flavones, substituted chalcones, substituted isoflavones, and mixtures thereof.

Flavonoid compounds can be obtained as extracts from natural sources such as plants. Examples of suitable flavonoid compounds include, but are not limited to, flavanone (unsubstituted), flavanonol (3′-hydroxy flavanone), pinocembrin (5,7-dihydroxy flavanone), pinostrobin (5-hydroxyl-7-methoxy flavanone), liquiritigenin (7,4′-dihydroxyflavanone), liquiritin (4′-glucoside-7,4′-dihydroxyflavanone), butin (7,3′,4′-trihydroxy flavanone), sakuranetin (5,4′-dihydroxy-7-methoxy flavanone), sakuranin (5-glucoside-5,4′-dihydroxy-7-methoxy flavanone), isosakuranetin (5,7-dihydroxy-4′-methoxy flavanone), poncirin (7-rhamnoglucoside-5,7-dihydroxy-4′-methoxy flavanone), naringenin (5,7,4′-trihydroxy flavanone), naringin (7-rhamnoglucoside-5,7,4′-trihydroxy flavanone), hesperitin (5,7,3′-trihydroxy-4′-methoxy flavanone), hesperidin (7-rhamnoglucoside-5,7,3′-trihydroxy-4′-methoxy flavanone), flavone (unsubstituted), chrysin (5,7-hydroxy flavone), toringin (5-glucoside-5,7-hydroxy flavone), apigenin (5,7,4′-trihydroxy flavone), apiin (7-apio-glucoside-5,7,4′-trihydroxy flavone), cosmosiin (7-glucoside-5,7,4′-trihydroxy flavone), acacetin (5,7,-dihydroxy-4′-methoxy flavone), fortunellin (7-rhamnoglucoside-5,7,-dihydroxy-4′-methoxy flavone), baicalein (5,6,7-trihydroxy flavone), baicalin (7-glucuronide-5,6,7-trihydroxy flavone), scutellarin (7-glucuronide-5,6,7,4′-tetrahydroxy flavone), diosmetin (5,7,3′-trihydroxy-4′-methoxy flavone), diosmin (7-rhamnoglucoside-5,7,3′-trihydroxy-4′-methoxy flavone), galangin (3,5,7-trihydoxy flavone), quercetin (3,5,7,3′,4′-pentahydroxy flavone), quercitrin (3-rhamnoside-3,5,7,3′,4′-pentahydroxy flavone), rutin (3-rhamnoglucoside-3,5,7,3′,4′-pentahydroxy flavone), rhamnetin (3,5,3′,4′-tetrahydroxy-7-methoxy flavone), xanthorhamnin (3-rhamnoside-3,5,3′,4′-tetrahydroxy-7-methoxy flavone), myricetin (5,7,3′,4′,5′-pentahydroxy flavonol), myricitrin (3-rhamnoside-5,7,3′,4′,5′-pentahydroxy flavonol), biflavones like fukugetin; ginkgetin and bilobetin, isoflavone, chalcone, all isomers of above substituted flavones, and mixtures thereof.

The flavonoid compounds useful herein can be synthetic materials derived or modified from naturally sourced material. With these chemical modifications, the flavonoid compounds can become more applicable to skin care compositions with improved solubility or compatibility with other composition components. Preferred modified flavonoid compounds are glycosylated, alkylated or acylated from naturally sourced material.

A particularly useful group of glycoside flavonoids herein are those selected from the general structural formula (III) 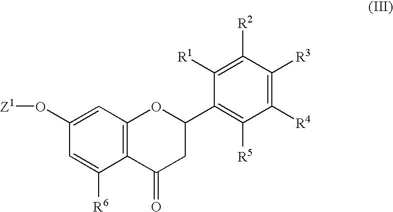
wherein R¹-R⁶ are independently selected from the group consisting of H, OH, alkoxy and hydroxyalkoxy, wherein the alkoxy or hydroxyalkoxy groups are branched or unbranched and have 1-18 carbon atoms, and wherein Z¹ is selected from the group consisting of mono- and oligoglycoside radicals. Z¹ is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

Another particularly useful group of glycoside flavonoids herein are those selected from the general structural formula (IV) 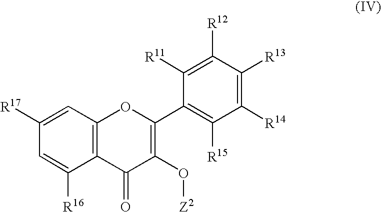
wherein R¹¹-R¹⁷ are independently selected from the group consisting of H, OH, alkoxy and hydroxyalkoxy, wherein the alkoxy or hydroxyalkoxy groups are branched or unbranched and have 1-18 carbon atoms, and wherein Z² is selected from the group consisting of mono- and oligoglycoside radicals. Z² is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

In one particularly preferred embodiment of the present invention, the glycoside flavonoid is selected from the group consisting of glucosyl hesperidin, glucosyl rutin, glucosyl myricitrin, glucosyl isoquercitrin, glucosyl quercitrin, methyl hesperidin, and mixtures thereof. These glucoside flavonoid compounds can be obtained by bio-chemical methods from related natural flavonoid compounds. The glucosyl group(s) can be connected to one or more hydroxides of the original substances.

A representative formula of glucosyl rutin is as follows:

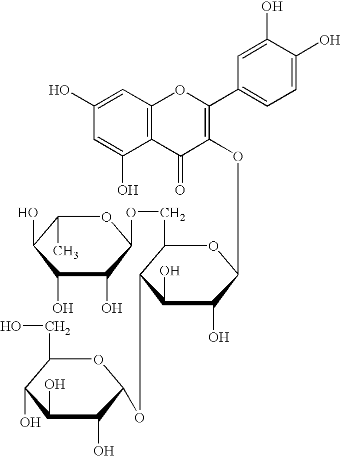

A representative formula of glucosyl hesperidin is as follows:

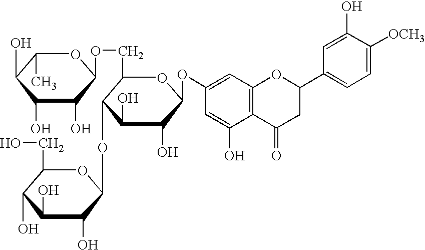

Alkylated flavonoid compounds useful herein are alkoxy or hydroxyalkoxy flavonoids that are usually derived from chemical modification of common natural flavonoids. Examples of alkylated flavonoid compounds useful herein are as follows. The formulae are merely representative, it is possible that the alkyl or hydroxyalkyl group is connected to other existing hydroxyl groups.

A representative formula of troxerutin (3′,4′,7-tri-hydroxyethoxyl-rutin) is as follows:

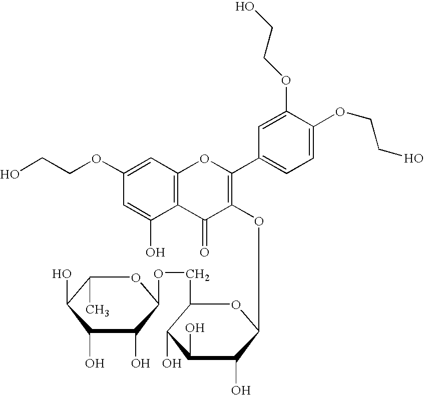

A representative formula of monoxerutin (7-hydroxyethoxyl rutin) is as follows:

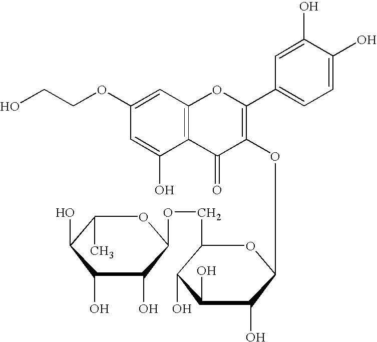

Another useful alkylated flavonoid compound, methyl hesperidin, has the general structural formula (V) 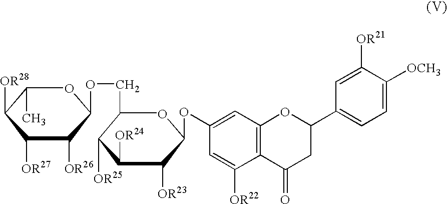
wherein R²¹-R²⁸ are independently selected from the group consisting of H or methyl, wherein at least one of R²¹-R²⁸ is methyl.

Another useful group of glycoside flavonoids herein is chalcone, which can be obtained by isomerization from any flavanone. Chalcones are highly useful in this invention due to their improved solubility which makes it easier to formulate into skin care compositions.

A representative chalcone derived from hesperidin is as follows:

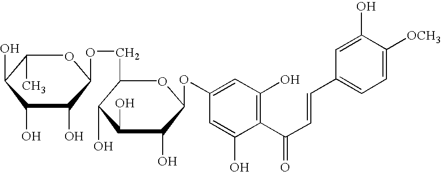

Another representative chalcone derived from hesperitin is as follows:

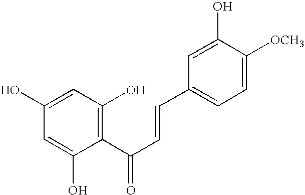

Commercially available flavonoid compounds include hesperidin, methylhesperidin, and rutin available from Alps Pharmaceutical Industry Co. Ltd. (Japan); and glucosyl hesperidin and glucosyl rutin available from Hayashibara Biochemical Laboratories, Inc. (Japan) and Toyo Sugar Refining Co. Ltd. (Japan).

Vitamin B3 Compound

The present device preferably further comprises a vitamin B3 compound. The vitamin B3 compound is used at a level of from about 0.01% to about 15%, preferably from about 0.1% to about 15%, more preferably from about 0.5% to about 10%, by weight of the liquid composition, gel sheet, or coating composition of the present device, as described hereinafter. Vitamin B3 compounds are known to provide, by itself, a precursor for nicotinamide adenine dinucleotide phosphate (NADP) family and its reduced form (NADPH) family of coenzymes, which enhance many metabolic enzyme reactions on the skin. Vitamin B3 compounds are also known to provide reduction in trans-epidermal water loss and excess dermal glycosaminoglycans, which are indicators for skin barrier properties.

It has been surprisingly found that, by the combined use of a flavonoid compound and a vitamin B3 compound, a composition providing synergistic skin treatment benefit over the single use of either active agent is obtained. Skin treatment benefit is particularly seen in skin lightening benefit and anti-aging benefit.

Without being bound by theory, it is believed that the flavonoid compound enhances the transportation of vitamin B3 compound. Flavonoid compounds have relatively good affinity with the cell membrane lipid bilayer, while vitamin B3 compounds have less affinity due to its generally hydrophilic structure. By the effective transportation of the 2 types of actives into the skin cells, it is believed that the 2 types of actives provide skin treatment benefits via different mechanisms in the dermis, thereby providing synergistic benefit to the skin.

Vitamin B₃ compounds useful herein include, for example, those having the formula: 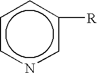
wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.
Vitamin B6 Compound

The present device preferably further comprises a vitamin B6 compound. The vitamin B6 compound is used at a level of from about 0.001% to about 15%, preferably from about 0.01% to about 10% by weight of the composition, more preferably from about 0.01% to about 5%, by weight of the liquid composition, gel sheet, or coating composition of the present device, as described hereinafter. Vitamin B6 compounds are known to provide, by itself, a coenzyme for synthesis of amino acids and nucleic acids, thereby enhancing anabolic activity, such as collagen synthesis, of skin cells. Improved collagen structure is known to provide good skin tone, and lightening appearance of the skin.

It has been surprisingly found that, by the combined use of a vitamin B6 compound in addition to a flavonoid compound and a vitamin B3 compound, a composition providing significant skin treatment benefit is obtained. Skin treatment benefit is particularly seen in skin lightening benefit and anti-aging benefit.

Without being bound by theory, it is believed that the three types of actives provide an integrated effect to the skin via different mechanisms in the dermis, thereby providing synergistic benefit to the skin.

Vitamin B6 compounds useful herein include pyridoxine; esters of pyridoxine such as pyridoxine tripahnitate, pyridoxine dipalmitate, and pyridoxine dioctanoate; amines of pyridoxine such as pyridoxamine; salts of pyridoxine such as pyridoxine HCl; and derivatives thereof such as pyridoxamine, pyridoxal, pyridoxal phosphate, and pyridoxic acid. Particularly useful vitamin B6 compounds are selected from the group consisting of pyridoxine, esters of pyridoxine and salts of pyridoxine. The vitamin B6 compound can be synthetic or natural in origin and can be used as an essentially pure compound or mixtures of compounds (e.g., extracts from natural sources or mixtures of synthetic materials). As used herein, “vitamin B6” includes isomers and 6 tautomers of such. Commercially available vitamin B6 compound useful herein include, for example, pyridoxine HCl available from DSM, pyridoxine dipalmitate with tradename NIKKOL DP and pyridoxine dioctanoate with tradename NIKKOL DK available from Nikko Chemicals Co. Ltd.

Preformed Substrate and Form of Device

The present device is based on a preformed substrate that is capable of retaining its structure and character on the skin surface for at least the time for which the product is designed to treat the skin. Generally speaking, substrate based skin care devices are kept intact with the skin for at least 5 minutes. The substrate based skin care device according to the present invention are generally of a size such that each surface has an area of from about 0.25 cm² to about 1,000 cm², preferably from about 1 cm² to about 300 cm². Surface area refers to that of a flat plane having the same boundary as the surface i.e. ignoring any surface texturing present. The substrate based skin care device herein can be, for example, square, circular, semicircular, rectangular, oval, rings, crescents, teardrops or other more complex shapes which may be composites of these, for covering areas such as the eye area, eye lids, the nose, the mouth area, the forehead, the chin, the entire contour of the face, neck, or combinations thereof.

The preformed substrate may be provided in various form and composition.

In one preferred embodiment, the device is in the form of what is typically called a treatment mask, wherein the substrate is water-insoluble, and wherein the remainder of the device forms a liquid composition; the liquid composition being 100% to 2000% by weight of the water-insoluble substrate. In this embodiment, the liquid composition and substrate are substantially inert of each other. For convenience, this embodiment is hereinafter called “mask”, and the terms “water-insoluble substrate” and “liquid composition” herein are specifically directed to the mask embodiment.

In another preferred embodiment, the device is in the form of what is typically called a patch or gel, wherein the substrate is a gel sheet comprising a gelling agent and a hydrophilic solvent for the gelling agent, and wherein the gel sheet encompasses the flavonoid compound. For convenience, this embodiment is hereinafter called “gel sheet”, and the terms “coating composition” and “gelling agent” herein are specifically directed to the gel sheet embodiment.

In the gel sheet embodiment, the substrate itself releases the flavonoid compound when in contact with the skin. In a particularly preferred embodiment, the device further comprises a discrete coating composition in addition to the gel sheet, wherein the coating composition also comprises the flavonoid compound. The coating composition may be included in the same package as the gel sheet to reach equilibrium in the package. By “discrete” coating composition is meant one that is applied to the gel sheet as a distinctly different composition, in particular one having a different chemical constitution which is separately prepared from the gel sheet and is laid down as a separate layer, before, after or at the same time as the formation of the gel sheet. The coating composition allows more efficient delivery of the flavonoid compound and other skin active agents to the skin and affords greater formulation flexibility. The coating composition preferably comprises the flavonoid compound, i.e., at least one skin active agent in common. In this way, whilst the coating composition can rapidly provide the skin active agent to the target area, the gel sheet can act as a reservoir for the skin active agent or inhibit the gel sheet from absorbing the flavonoid compound and other skin active agents from the coating composition.

The gel sheet embodiment may further comprise a water-insoluble substrate, such as in the form of a film, for adding strength or occlusivity.

Liquid Medium

The device of the present invention comprises a liquid medium which is liquid at 25° C. for delivering the flavonoid compound and other optional skin active agents to the skin via the substrate, regardless of the form of the composition. The liquid medium is typically water or a water solution. When the preferred present device comprising water is applied to a certain area of the skin, the temperature of the certain skin surface significantly drops due to the water. In the present invention, however, the existence of the flavonoid compound in the composition alleviates temperature drop of the skin surface. Without being bound by theory, such alleviation of temperature drop is believed to be due to the circulation enhancing ability of the flavonoid compounds. In that penetration of active agents through the skin is decelerated as temperature of the skin surface is decreased, alleviation of temperature drop is advantageous.

For the mask embodiment, the liquid medium is used as a carrier for the liquid composition. For the gel sheet embodiment, the liquid medium is used as the hydrophilic solvent for the gelling agent, and also a carrier for the coating composition.

The level and species of the liquid medium are selected according to the compatibility with other components, and other desired characteristic of the product. Liquid mediums useful in the present invention include water and water solutions of lower alkyl alcohols. Lower alkyl alcohols useful herein are monohydric alcohols having 1 to 6 carbons, more preferably ethanol. Deionized water is preferably used. Water from natural sources including mineral cations can also be used, depending on the desired characteristic of the product.

The liquid medium preferably contains a water soluble humectant to provide moisturizing benefit to the skin. Water soluble humectants useful herein include polyhydric alcohols such as glycerin, diglycerin, propylene glycol, dipropylene glycol, butylene glycol, hexylene glycol, sorbitol, ethoxylated glucose, 1,2-hexane diol, hexanetriol, erythritol, trehalose, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein also include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

When used as the hydrophilic solvent for the gelling agent of the gel sheet, the liquid medium is selected so that a gel phase is made with the gelling agent at room temperature. The amount and type of solvent is determined to provide a stable gel phase with the gelling agent, in view of the desired mechanical properties, particularly gel strength and flexibility, and also in view of the desired characteristic of the obtained product. Preferably, the solvent further acts as a plasticiser or softener for the gel sheet. The gel sheet preferably comprises from about 10% to about 99.5% of water, more preferably from about 20% to about 95%, and yet more preferably from about 30% to about 90% of water. Still preferably, the gel sheet further comprises from about 1.0% to about 50%, preferably from about 5% to about 45%, more preferably from about 10% to about 40% of polyhydric alcohol.

Water-Insoluble Substrate

The water-insoluble substrate as may be used herein is the implement or vehicle for delivering the liquid composition to the skin for the present mask embodiment. By “Water insoluble”, it is meant that the substrate does not dissolve in or readily break apart upon immersion in water.

A wide variety of materials can be used as the water-insoluble substrate. The following nonlimiting characteristics are desirable: (i) sufficient wet strength for use, (ii) sufficient thickness, and (iii) appropriate size.

Nonlimiting examples of suitable water-insoluble substrates which meet the above criteria include nonwoven substrates, woven substrates, hydroentangled substrates, air entangled substrates, natural sponges, synthetic sponges, polymeric netted meshes, and the like. Preferred embodiments employ nonwoven substrates since they are economical and readily available in a variety of materials. By “nonwoven”, it is meant that the layer is comprised of fibers which are not woven into a fabric but rather are formed into a sheet, mat, or pad layer.

The water-insoluble substrates may be comprised of a variety of materials both natural and synthetic. Nonlimiting examples of natural materials useful in the present invention include: silk fibers; keratin fibers such as wool fibers and camel hair fibers; and cellulose fibers such as wood pulp fibers, cotton fibers, hemp fibers, jute fibers, and flax fibers. Nonlimiting examples of synthetic materials useful in the present invention include: acetate fibers; acrylic fibers; cellulose ester fibers; polyamide fibers; polyester fibers such as polyethylene terephthalate fibers; polyolefin fibers such as polypropylene fibers and polyethylene fibers; polyvinyl alcohol fibers; rayon fibers; and polyurethane foam.

Water-insoluble substrates useful in the present invention can also be obtained from a wide variety of commercial sources. Nonlimiting examples of suitable nonwoven substrates useful herein include: WALKISOFT®, a cellulose substrate available from Walkisoft U.S.A.; NOVONET® 149-801 and 149-191, a substrate containing about 69% rayon, about 25% polypropylene, and about 6% cotton, available from Veratec, Inc. Walpole, Mass.; KEYBAK® 951V and 1368, a substrate containing about 75% rayon and about 25% acrylic fibers, available from PGI/Chicopee, Dayton, N.J.; RMT-90, a 3-layer substrate having a pulp layer as an inner layer with outer layers respectively made of the combination of rayon and polyester, and RFP-90, a 3-layer substrate having a combined PP layer as an inner layer with outer layers of rayon, R-80, a single layer substrate containing 100% rayon, all available from Daiwabo K.K.

The water-insoluble substrate is advantageous for providing a device that corresponds to the shape of the face. In one highly preferred embodiment, the water-insoluble substrate is so configured to cover substantially the whole area of the facial skin with areas of the eyes and nostrils opened. In another highly preferred embodiment, the water-insoluble substrate is so configured to cover substantially the whole area of the facial skin, and is made of two pieces, the first piece covering the upper area of the face, i.e. the nose and thereabove, and the second piece covering the lower area of the face, i.e. the upper lip, cheeks and thereunder. In another preferred embodiment, the water-insoluble substrate is so configured to match the area of a particular part of the face, such as the nose, cheekbone, chin, forehead, neck, or combinations thereof. In another highly preferred embodiment, the water-insoluble substrate is so configured to have ears, pulls, or rings for facilitating placement and/or removal of the composition on the skin.

The water-insoluble substrate is flexible enough such that, when soaked with the liquid composition, readily fits along the skin, yet strong enough so that it does not easily tear or crumble upon use. Preferably, the water-insoluble substrate has a thickness of from about 100 μm to about 1 cm, more preferably from about 300 μm to about 3 mm, depending on the material for making the water-insoluble substrate, and use and characteristic of the product.

Water-insoluble substrate materials particularly useful herein include those which are of hydrophilic nature, thereby capable of absorbing a larger quantity of the liquid composition. The water-insoluble substrate can be made solely of hydrophilic material, or made of a mixture of hydrophilic material and hydrophobic material. The water-insoluble substrates of the present invention can consist of a single layer or multiple layers. In one preferred embodiment, the water-insoluble substrate is made of at least partially by hydrophilic materials selected from cotton, pulp, rayon, and mixtures thereof. By partially, it is meant to encompass following situations: where one layer of a hydrophilic material is used for a single layered substrate; where at least one layer of a hydrophilic material is used for a multiple layered substrate; where one layer of a mixture of the hydrophilic material and another material is used for a single layered substrate; and where at least one layer of a mixture of the hydrophilic material and another material is used for a multiple layered substrate.

When the water-insoluble substrate consists of multiple layers, it is preferred that at least the layer facing the skin is that of hydrophilic nature, thereby capable of absorbing a larger quantity of the liquid composition. When the water-insoluble substrate consists of multiple layers, the substrates can include films and other nonfibrous materials. In one embodiment, the water-insoluble substrate may also be laminated with polymeric film on the substrate, coating the substrate, or heat sealing the substrate. The resulting substrate with the laminated polymeric film, coating or heat sealing comprises an occluded side on one side of the substrate, which faces away from the skin, and a skin facing side that is positioned on the skin surface. By having a substrate with an occluded side, the substrate acquires low air permeability. By “low air permeability” it is meant that the side of the substrate having the film, coating or heat sealing allows very little air to enter into the substrate and very little vapor to escape from the substrate. Preferably the air permeability is less than about 5 mg/cm²/min.

A water-insoluble substrate comprising an occluded side significantly increases penetration of skin active agents into the skin compared to a water-insoluble substrate without an occluded side. Additionally, the substrate based skin care device of the present invention utilizes water tension to adhere to the skin surface rather than strong adhesives on the skin facing side. The absence of a strong adhesive between the substrate and the skin surface, as utilized by the present invention, removes the physical barrier resulting from the strong adhesive and promotes the penetration of the oily components and other skin active agents. The resulting environment between the skin and the water-insoluble substrate promotes the penetration of the skin active agents into the skin.

Suitable polymeric film includes polyethylene, polypropylene, polyethylene terephtalate, polyamides, polyesters, nylons, blends thereof, or any other cosmetically acceptable polymeric films. Suitable coatings include any materials known in the art that impart low air permeability to the water-insoluble substrate and are cosmetically acceptable. Heat-sealing the water-insoluble substrate may be accomplished by any method known in the art to impart low air permeability to the substrate.

In one embodiment, the water-insoluble substrate comprises a layer having a gradient of hydrophilic properties, i.e., having a gradient of distribution of hydrophobic materials and hydrophilic materials. In this water-insoluble substrate, it is preferred that the substrate has a high distribution of hydrophilic materials at the skin facing side, and a high distribution of hydrophobic materials at the opposite side. This structure allows for moisture to be move from the hydrophobic side of the substrate to the hydrophilic skin facing side of the substrate to the skin surface. This water-insoluble substrate can be a single layered substrate, can further comprise other layers, or can be laminated with a polymeric film.

Liquid Composition

The liquid composition of the present mask embodiment impregnates, coats or is otherwise in contact with the water-insoluble substrate described hereinbefore.

The liquid composition herein comprises:
(a) a water-soluble thickening agent which provides the liquid composition a viscosity of from about 10 mPa·s to about 600,000 mPa·s; and
(b) water.

The amount of liquid composition associated with any composition will vary depending upon the desired characteristics of the finished mask embodiment device, but should be at least an amount sufficient to result in delivering the flavonoid compound to the skin surface. Preferably, the water-insoluble substrate remains saturated with the liquid composition during the interval between the saturation of the water-insoluble substrate with the liquid composition and application. To that desired end, the liquid composition will therefore preferably represent from about 100% to about 2000%, more preferably from about 200% to about 1500%, by weight of the water-insoluble substrate. The amount of liquid composition to be used will depend on the absorbing capability of the water-insoluble substrate, and the desired characteristic of the composition.

The liquid compositions used for the mask of the present invention have a viscosity in the range of from about 1000 mPa·s to about 600,000 mPa·s, preferably from about 2000 mPa·s to about 300,000 mPa·s, more preferably from about 3000 mPa·s to about 100,000 mPa·s, as measured by a Brookfield Digital Viscometer, Model DV-II+ Version 3.2 according to the operating instructions set forth in Manual No. M/92-161-H895. Such viscosity is believed to be suitable for suspending the skin tone changing agents in the liquid composition in an effective manner, as well as effectively depositing the skin tone changing agents and other benefit agents to the skin.

Gelling Agent

The gel sheet embodiment of the present invention comprises a gelling agent that provides a stable solid structure at normal storage temperature. Mixtures of gelling agents can be used. The type and amount of gelling agent to be included in the gel sheet is selected according to the desired characteristic and purpose of the product, depending on the properties of the gelling agent. Generally, more gelling agent will provide a more rigid gel sheet. Many types of gelling agents are known in the art, including polymeric gellants and particulate based gellants such as various types of clays or other silicate based materials. Highly preferred herein are polymeric gelling agents that form 3-dimensional gel networks in combination with water. The gel sheet preferably comprises from about 0.5% to about 20%, more preferably from about 1% to about 10%, of gelling agent.

The gelling agents herein may be water soluble or water insoluble, and selected according to the solvent. When the solvent is water or water-based, preferably the gelling agent is water soluble. Water insoluble polymeric gellants such as silicone materials e.g. organopolysiloxane resins, or block co-polymer thermoplastic elastomers, may be used in an appropriate solvent.

The water-soluble polymeric gellants for use in the present invention are selected from synthetic or natural polymers, and mixtures thereof. Preferred polymers for use herein are natural polymers, including gelatin, polysaccharides, and mixtures thereof. Preferred are polysaccharides. The term “polysaccharide” herein means a naturally occurring or synthetically produced, linear, branched or cross-linked polymer of monosaccharide units, which swells when dispersed in water at low concentrations and thickens the aqueous phase.

The polysaccharides for use in the gel sheets herein are preferably selected from red seaweed polysaccharides; glucomannans; galactomannans; fermentation poly-saccharides, or derivatives thereof; brown seaweed polysaccharides; extracts of marine invertebrates; starch, or derivatives thereof; natural fruit extracts; plant fiber derivatives; kelp; natural plant exudates; and resinous gums; or mixtures thereof. The total polysaccharide level is controlled so that the skin active agents, including the flavonoid compound, of the gel sheet are not as tightly bound within the gel network and are available for diffusion.

When gelatin is used in the gel sheets herein, a high-molecular weight gelatin is combined with a low-molecular weight one to control the solubility. A gelatin having a low molecular weight of 20,000 or less has weaker gelling ability when used as the sole gelling agent.

Brown seaweed polysaccharides are isolated by extraction from various species of Phaebophyceae. Suitable brown seaweed polysaccharides for use herein include algin, alginic acid, ammonium alginate, calcium alginate, potassium alginate, sodium alginate, propylene glycol alginate, and mixtures thereof.

Red seaweed polysaccharides are isolated from marine plant species belonging to the class of Rhodophyceae. Red seaweed polysaccharides provide mechanical strength to the gel sheet. Suitable red seaweed polysaccharides for use in the present invention include agar known in the industry under the (CTFA) trade designation as agar agar flake derived from various Gelidium plant species or closely related red algae commercially available as “Agar Agar 100” or “Agar Agar 150” from TIC Gums (Belcamp, Md., USA) or “Agar Agar K-100” from Gumix International Inc. (Fort Lee, N.J., USA); agarose commercially available as “Sea Plaque®” from FMC (Philadelphia, Pa., USA) and “Agarose Type 1-b” from Sigma—Aldrich Co. Ltd. (Poole, UK); carrageenan, comprising the fractions lambda-, iota- and kappa- which are the water extracts obtained from various members of the Gigartinaceae or Solieriaceae families, known in the industry under the (CTFA) trade designation as chondrus, commercially available as “Gelcarin® LA”, “Seakem® 3/LCM”, or “Viscarin® XLV”, all from FMC (Philadelphia, Pa., USA); and furcellaran commercially available from Gum Technology Corporation (Tucson, Ariz., USA) and Continental Colloids Inc. (Chicago, Ill., USA), or mixtures thereof. Preferably, the red seaweed polysaccharide for use herein is selected from agar, agarose, kappa-carrageenan and furcellaran, or mixtures thereof.

Glucomannans are polysaccharides which comprise an essentially linear backbone of glucose and mannose residues. Glucomannans have short side branches attached to the linear backbone and acetyl groups are randomly present at the C-6 position of a sugar unit. The acetyl groups are generally found on one per six sugar units to one per twenty sugar units. Suitable glucomannans or derivatives thereof for use herein have a ratio of mannose to glucose of from about 0.2 to about 3. Preferred glucomannans for use herein include konjac mannan, which is the generic name for the flour formed from grinding the tuber root of the Amorphophallus konjac plant (elephant yam), commercially available under the trade name “Nutricol® konjac flour” from FMC (Philadelphia, Pa., USA); and deacetylated konjac mannan; or mixtures thereof.

Galactomannans are vegetable reserve polysaccharides which occur in the endosperm cells of numerous seeds of Leguminosae. The collective term “galactomannan” comprises all polysaccharides which are built up of galactose and mannose residues. Galactomannans comprise a linear backbone of (1→4)-linked β-D-mannopyranosyl units. To these rings are attached as branches, isolated galactopyranose residues by α-(1,6)-glucoside bonds. Galactomannans may in addition also contain minor amounts of other sugar residues. Suitable galactomannans for use herein are fenugreek gum; lucern; clover; locust bean gum known for example in the industry under the (CTFA) trade designation as carob bean gum, commercially available as “Seagul L” from FMC (Philadelphia, Pa., USA); tara gum commercially available from Starlight Products (Rouen, France) or Bunge Foods (Atlanta, Ga., USA); guar gum derived from the ground endosperms of Cyamopsis tetragonolobus, commercially available as “Burtonite V7E” from TIC Gums (Belcamp, Md., USA), “Jaguar C” from Rhone-Poulenc (Marietta, Ga., USA), or “Supercol” from Aqualon (Wilmington, Del., USA); and cassia gum commercially available from Starlight Products (Rouen, France), or mixtures thereof. Preferably, the galactomannans for use herein have an average one of every 1 to about 5 mannosyl units substituted with a (1→6)-linked-α-D-galactopyranosyl unit and are selected from guar gum, locust bean gum and cassia gum, or mixtures thereof.

Fermentation polysaccharides are polysaccharides which are commercially produced by the fermentation of micro-organisms in a medium containing a carbon and nitrogen source, buffering agent, and trace elements. Suitable fermentation poly-saccharides or derivatives thereof, for use in the present invention include gellan gum known in the industry under the (CTFA) trade designation as gum gellan, a high molecular weight hetero polysaccharide gum produced by a pure-culture fermentation of a carbohydrate with Pseudomonas elodea, commercially available as “Kelcogel” from Kelco (San Diego, Calif., USA); xanthan gum which is a high molecular weight hetero polysaccharide gum produced by a pure-culture fermentation of a carbohydrate with Xanthomonas campestris, known in the industry under the (CTFA) trade designation as xanthan, commercially available for example as “Keltrol CG 1000/BT/F/GM/RD/SF/T/TF”, from Calgon (Pittsburgh, Pa., USA), or “Kelzan” from Kelco (San Diego, Calif., USA); natto gum; pullulan; rhamsan gum; curdlan; succinoglycan; welan gum; dextran, commercially available as “Sephadex G-25” from Pharmacia Fine Chemicals (Piscataway, N.J., USA) and derivatives thereof; and sclerotium gum, commercially available as “Amigel” from Alban Muller International (Montreil, France), or mixtures thereof. Preferred fermentation polysaccharides or derivatives thereof are selected from gellan gum and xanthan gum, or mixtures thereof. More preferably the fermentation polysaccharide or derivative thereof is xanthan gum.

Extracts of marine invertebrates can also be used. Polysaccharides derived from marine invertebrates, specifically the exoskeleton of such invertebrates, consist chiefly of N-acetyl-D-glucosamine residues. Examples of such polysaccharides suitable for use herein include chitosan, commercially available for example as “Marine Dew” from Ajinomoto (Teakneck, N.J., USA); and hydroxypropyl chitosan commercially available for example as “HPCH Liquid” from Ichimaru Pharcos (Yamagata Gun Gifu-Pref, Japan) and derivatives; or mixtures thereof.

Starches are polysaccharides which consist of various proportions of two glucose polymers, amylose and amylopectin. Suitable materials for use herein include starch, amylopectin and dextrin, commercially available as “Nadex 360” from National Starch (Bridgewater, N.J., USA), and derivatives or mixtures thereof. Examples of natural fruit extracts suitable for use herein include pectin, arabian and mixtures thereof. A suitable example of a plant fiber derivative for use herein is cellulose. Suitable polysaccharides obtained from natural plant exudates for use herein include karaya, tragacanth, arabic, tamarind, and ghatty gums, or mixtures thereof. Examples of resinous gums suitable for use herein include shellac gum, which is obtained from the resinous secretion of the insect Laccifer (Tachardia) lacca, damar gum; copal gum and rosin gum; or mixtures thereof.

Natural and synthetic polymeric gelling agents that form gels in combination with other substances, may also be used as a gelling agent in combination with other thermo-forming gelling agents, so long as the gel phase forming can been synchronized. They may be chemically cross linked. Some gelling agents form gels in combination with substances such as sugar, alcohol, or mono- or multi-valent salts. Mono- or multi-valent salts may additionally act as gel strengthening agents imparting added strength to the gel sheets herein. Suitable cations for such salts can be selected from potassium, sodium, ammonium, zinc, aluminium, calcium and magnesium ions, or mixtures thereof. Suitable anions associated with the aforementioned cations may be selected from chloride, citrates, sulfate, carbonate, borate and phosphate anions, or mixtures thereof.

Physical cross linking refers to polymers having cross links which are not chemical covalent bonds but are of a physical nature such that there are areas having high crystallinity or areas having a high glass transition temperature. Such cross linked polymers may also be used. Preferably, the polymer is chemically cross linked thermally. In addition when chemical cross links are formed in the system, a poly-functional cross linker and/or a free radical initiator may be present in the premix to initiate the cross linking upon irradiation.

Preferably, the gel sheets herein comprise a mixture of water-soluble polymeric gelling agents of natural origin. A preferred water-soluble polymeric gelling agent mixture herein may comprise a polysaccharide and a non-ionic water-soluble polymer or, alternatively, it may comprise two polysaccharides. More preferably, the water-soluble polymeric gel forming agent is a polysaccharide mixture, wherein the polysaccharide mixture comprises (1) at least one red seaweed polysaccharide; brown seaweed polysaccharide; or mixtures thereof; and (2) at least one fermentation polysaccharide; galactomannan; glucomannan; natural plant exudate; or natural fruit extract; and derivatives or mixtures thereof. Even more preferably, the water-soluble polymeric gel forming agent of the gel sheets of the present invention is a polysaccharide mixture comprising (1) at least one red seaweed polysaccharide; and (2) at least one fermentation polysaccharide; glucomannan; or galactomannan; and derivatives or mixtures thereof.

In a preferred embodiment, the water-soluble polymeric gel forming agent of the present invention is a polysaccharide mixture, comprising a red seaweed polysaccharide and a glucomannan or a galactomannan. The ratio of red seaweed polysaccharide to glucomannan or galactomannan in the polysaccharide mixture is preferably from about 20:1 to about 1:5 and more preferably from about 10:1 to about 1:2. Without being limited by theory, it is believed that gel compositions herein form 3-dimensional networks or matrices which bind or encapsulate other ingredients of the composition.

Water Soluble Thickening Agent

The liquid compositions of the mask embodiment comprise a water-soluble thickening agent. Water soluble thickening agents may also be used for the gel sheet and/or coating composition. The water soluble thickening agents herein are water soluble or water miscible polymers, have the ability to increase the viscosity of the composition, and are compatible with other components used in the composition. The water soluble thickening agent is selected so that the liquid composition of the present composition has the desired viscosity of from about 10 mPa·s to about 600,000 mPa·s, preferably from about 10 mPa·s to about 100,000 mPa·s, more preferably from about 100 mPa·s to about 30,000 mPa·s. The water soluble thickening agents are included, by weight of the liquid composition, at a level preferably from about 0.01% to about 5%, more preferably from about 0.1% to about 2%.

Water soluble thickening agents useful herein include anionic polymers and nonionic polymers. The water soluble thickening agents useful herein include, for example, acrylic polymers, polyalkylene glycol polymers having a molecular weight of more than about 10000, celluloses and derivatives there of such as hydroxyethyl cellulose, polyvinylpyrrolidone, polyvinyl alcohol, gums such as guar gum and xanthan gum, carragenan, pectin, agar, quince seed (Cydonia oblonga Mill), starch (rice, corn, potato, wheat), algae colloids (algae extract), dextran, succinoglucan, pulleran, carboxymethyl starch, methylhydroxypropyl starch, sodium alginate, and alginic acid propylene glycol esters. Neutralizing agents may be included to neutralize the anionic thickening agents described hereinabove. Nonlimiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

Among the above polymers, highly preferred are those providing reduced undesirable polymer flakes when emulsified liquid compositions are dried on the skin. Such highly preferred polymers include, for example, acrylic polymers. Acrylic polymers useful herein include those comprising monomers selected from the group consisting of acrylic acid, salts of acrylic acid, derivatives of acrylic acid, methacrylic acid, salts of methacrylic acid, derivatives of methacrylic acid, and mixtures thereof. The derivatives include, for example, alkyl acrylate, acrylamide, alkyl metahcrylate, and methacrylamide. Such acrylic polymers include, for example, cross linked acrylic acid polymers with the CTFA name Carbomer, sodium polyacrylate, polyacrylamide, and acrylic acid/alkyl acrylate copolymers with the CTFA name Acrylates/C10-30 Alkyl Acrylate Crosspolymer. Commercially available acrylic polymers highly useful herein include, for example, polyacrylamide with tradename Sepigel 305 available SEPPIC Inc., and Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Oily Component

The liquid composition, gel sheet, or coating composition of the present invention may further comprise an oily component. Oily components useful herein can deliver skin conditioning benefits such as smoothness and softness to the skin. Oily components useful herein include, for example, fatty alcohols, silicone oils, mineral oil, petrolatum, C₇₋₄₀ straight and branched hydrocarbons such as isohexadecane, C₁₋₃₀ alcohol esters such as isopropyl isostearate, glycerides, alkylene glycol esters, propoxylated and ethoxylated derivatives, sugar ester such as sucrose polycottonseedate, vegetable oils such as coconut oil, hydrogenated vegetable oils, animal fats and oils, and C₄₋₂₀ alkyl ethers of polypropylene glycols, C₁₋₂₀ carboxylic acid esters of polypropylene glycols, and di-C₈₋₃₀ alkyl ethers. Hydrophobic nonionic surfactants, which are those being water-insoluble and having an HLB value of less than 10, can be used as oily components. Hydrophobic nonionic surfactants useful herein include, for example, cetearyl glucoside, steareth-2, laureth-4, sucrose cocate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Among the above oily components, highly preferred are fatty alcohols, in that they provide skin conditioning benefits, and also in that they can form gel networks with surfactants which provide increased viscosity, phase stability, and conditioning benefits such as slippery feel. The fatty alcohols useful herein are a saturated, linear or branched fatty alcohol, selected from the group consisting of a saturated, linear or branched C₁₂₋₃₀ fatty alcohols, a saturated, linear or branched C₁₂₋₃₀ diols, and mixtures thereof. Preferred fatty alcohols are cetyl alcohol, stearyl alcohol, and mixtures thereof.

Hydrophilic Surfactant

The liquid composition, gel sheet, or coating composition of the present invention may further comprise a hydrophilic surfactant for dispersing or emulsifying the oil component. Hydrophilic surfactants useful herein are those being water-soluble, and preferably have an HLB value of above 10. Hydrophilic surfactants useful herein include, for example, any cosmetically acceptable surfactants, i.e., nonionic surfactants, cationic surfactants, anionic surfactants, zwitterionic surfactants, amphoteric surfactants, and mixtures thereof. Among them, preferred herein are cosmetically acceptable nonionic surfactants in view of reduced skin irritation and conditioning benefits.

Hydrophilic nonionic surfactants useful herein include, for example, PEG-100 stearate, polysorbate-20, polysorbate-60, seteareth-21, isoceteth-20, and oleth-20, laureth-23, ceteareth-12, steareth-100, PEG 40 hydrogenated castor oil, PEG-60 hydrogenated castor oil, and mixtures thereof.

Additional Skin Active Agent

The liquid composition, gel sheet, or coating composition of the present invention may further comprise a safe and effective amount of an additional skin active agent. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sunscreening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 30%, preferably from about 0.001% to about 10% by weight of the liquid composition, gel sheet, or coating composition, of an additional skin active agent.

The type and amount of skin active agents are selected so that the inclusion of a specific agent does not affect the stability of the composition.

Skin lightening agents useful herein refer to active ingredients that improve hyperpigmentation as compared to pre-treatment. Useful skin lightening agents herein include ascorbic acid compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C50 available from DSM.

Other hydrophobic skin lightening agents useful herein include ascorbic acid derivatives such as ascorbyl tetraisopalmitate (for example, VC-IP available from Nikko Chemical), ascorbyl palmitate (for example available from DSM), ascorbyl dipalmitate (for example, NIKKOL CP available from Nikko Chemical); undecylenoyl-phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, and mixtures thereof.

Other skin active agents useful herein include those selected from the group consisting of panthenol, benzoyl peroxide, 3-hydroxy benzoic acid, farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

Additional Components

The devices hereof may further contain additional components such as are conventionally used in topical products, e.g., for providing aesthetic or functional benefit to the composition or skin, such as sensory benefits relating to appearance, smell, or feel, therapeutic benefits, or prophylactic benefits. It is to be understood that the above-described required materials may themselves provide such benefits.

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

Method of Use

The skin care device of the present invention is suitable for topical application on human body skin, particularly facial skin. The use of the present device provides an enhanced penetration of components included in the device, particularly by the specific skin active agents included in the device. The device of the present invention is particularly advantageous in delivering the flavonoid compound and other skin active agents that the skin is exposed to an abundant amount of such agents over a lengthy period of time. By use of a preformed substrate as a delivery means over a lengthy period, is believed to provide better distribution and deposition of such agents, and better penetration of those agents which are percutaneously deliverable. Further, the device of the present invention is also believed to provide emotional benefits to the user upon use, such as refreshing feel, and relaxation feel. The devices herein are applied to the skin from about 5 to about 45 minutes, preferably from about 15 to about 45 minutes.

Because the device of the present invention is easily dried via exposure to regular atmospheric conditions, the device must be housed in a hermetically sealed package during storage.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Examples 1-12

Example 1 through 12 are embodiments of masks using, as a water-insoluble substrate, 3.0 g of substrate RFP-90 available from Daiwabo cut and shaped and soaked with 20-30 g each of the liquid compositions specified below in Tables 1 and 2.

TABLE 1

| Components | Ex. 1 | Ex. 2 | Ex. 3 | Ex. 4 | Ex. 5 | Ex. 6 |
|---|---|---|---|---|---|---|
| Glucosyl Hesperidin *1 | | 0.2 | | 0.3 | 0.2 | |
| Glucosyl Rutin *2 | 0.2 | | 0.2 | | | 0.2 |
| Niacinamide *3 | 3.5 | 5.0 | 3.5 | 3.5 | 3.5 | 5.0 |
| Pyridoxine Dipalmitate | 0.1 | 0.2 | 0.2 | 0.2 | 0.2 | 0.2 |
| *4 | | | | | | |
| Isopropyl Isostearate | 0.5 | 0.5 | 0.5 | 1.0 | | |
| *5 | | | | | | |
| Sucrose Polycotton- | 0.25 | 0.25 | 0.25 | | | |
| seedate *6 | | | | | | |
| Cetyl Alcohol | 0.2 | 0.2 | 0.3 | 0.3 | | |
| Stearyl Alcohol | 0.2 | 0.3 | 0.3 | 0.3 | | |
| Isohexadecane *7 | | 0.5 | 1.0 | 1.0 | 2.0 | 2.0 |
| Dimethicone and | | 0.5 | 1.0 | 0.5 | 1.5 | 1.5 |
| Dimethiconol *8 | | | | | | |
| PEG-100 stearate *9 | 0.1 | 0.1 | 0.1 | 0.1 | | |
| Cetearyl Glucoside *10 | 0.1 | 0.1 | 0.1 | 0.15 | | |
| Polyacrylamide *11 | 1.2 | 1.2 | 1.2 | | | |
| Acrylates/C10-30 | | | | 0.25 | 0.36 | 0.36 |
| Alkyl Acrylates | | | | | | |
| Crosspolymer *12 | | | | | | |
| Glycerin | 3.0 | 3.0 | 3.0 | 3.0 | 3.0 | 3.0 |
| 1,3-Butylene Glycol | 5.0 | 8.0 | 5.0 | 5.0 | 5.0 | 8.0 |
| Polymethyl- | 1.0 | 1.0 | | | 2.0 | 2.0 |
| silsesquioxane *13 | | | | | | |
| Sodium Ascorbyl | 0.5 | | 0.2 | | 2.0 | |
| Phosphate *14 | | | | | | |
| Panthenol *15 | 0.5 | 1.0 | 0.5 | | | |
| Tocopherol Acetate *16 | 0.2 | 0.2 | 0.2 | 0.2 | 0.2 | 0.2 |
| Mulberry extract *17 | 0.5 | | | | 0.5 | 0.5 |
| Perfume | 0.05 | 0.1 | 0.1 | | 0.1 | 0.1 |
| Benzyl Alcohol | 0.25 | 0.25 | 0.25 | 0.25 | 0.25 | 0.25 |
| Methyl Paraben | 0.15 | 0.15 | 0.15 | 0.15 | 0.15 | 0.15 |
| Ethyl Paraben | 0.07 | 0.07 | 0.07 | 0.07 | | |
| Propyl Paraben | 0.03 | 0.03 | 0.03 | 0.03 | | |
| Disodium EDTA | 0.1 | 0.1 | 0.1 | 0.1 | 0.1 | 0.1 |

| | |
|---|---|
| NaOH | Adjust pH to 6-8 |
| Deionized Water | q.s. to 100% |

Definitions of Components
*1 Glucosyl Hesperidin: alpha-Ghesperidin PS-CC available from Toyo Sugar Refining.
*2 Glucosyl Rutin: alpha-GRutin available from Toyo Sugar Refining.
*3 Niacinamide: Niacinamide USP available from DSM
*4 Pyridoxine Dipalmitate: Nikkol DP available from Nikko Chemicals.
*5 Isopropyl Isostearate: Schercemol 318 available from Scher Chemicals Inc.
*6 Sucrose Polycottonseedate: available from Kobo Products Inc.
*7 Isohexadecane: Permethyl 101A available from Bayer Corp.
*8 Dimethicone and Dimethiconol: Dow corning Q2-1503 fluid available from Dow Chemicals.
*9 PEG-100 stearate: Myrj 59P available from Uniqema
*10 Cetearyl Glucoside: Emulgade PL 68/50 available from Cognis
*11 Polyacrylamide: Sepigel 305 available from SEPPIC Inc
*12 Acrylates/C10-30 Alkyl Acrylates Crosspolymer: Pemulen TR-2 available from B. F. Goodrich
*13 Polymethylsilsesquioxane: Tospearl 145A from GE Toshiba
*14 Sodium Ascorbyl Phosphate: Stay-C 50 available from DSM
*15 Panthenol: DL-Panthenol available from Roche
*16 Tocopherol Acetate: available from Eisai Co. Ltd.
*17 Mulberry extract: mulberry extract BG available from Maruzen Pharmaceuticals

Method of Preparation for Examples 1-6

The compositions above described can be made by any method known to the artisan. The compositions are suitably made as follows:
(1) All oily components and surfactants are mixed in a vessel, and heated to 75° C. or above in order to melt solid oily compounds if included.
(2) Water-soluble and heat-stable ingredients other than water soluble thickening polymers are dissolved in water in another vessel, and heated to 75° C. or above.
(3) The products of steps (1) and (2) are mixed until homogeneous.
(4) Water soluble thickening polymers are added to the product of step (3) and mixed until homogeneous.
(5) The product of step (4) is cooled down to 40° C. or below.
(6) Other water soluble ingredients like hesperidin and niacinamide, extract, etc. are dissolved into water, and added to above step (5) mixture.
(7) If included, other remaining components such as perfumes are added to the product of step (5).
(8) The water-insoluble substrate is folded and placed in an aluminium pouch per one unit.

(9) The emulsified liquid composition thus obtained at step (7) is poured into the aluminum pouch containing the water-insoluble substrate or preformed gel patch, and hermetically sealed.

TABLE 2

| Components | Ex. 7 | Ex. 8 | Ex. 9 | Ex. 10 | Ex. 11 | Ex. 12 |
|---|---|---|---|---|---|---|
| Glucosyl Hesperidin | 0.2 | 0.2 | 0.2 | 0.2 | 0.2 | 0.1 |
| *1 | | | | | | |
| Niacinamide *2 | 3.5 | 5 | 3.5 | 5.0 | 3.5 | 2 |
| Pyridoxine Hydro- | 0.1 | 0.1 | 0.1 | 0.1 | 0.1 | 0.1 |
| chloride *3 | | | | | | |
| Xanthan Gum *4 | 0.6 | 0.6 | 0.4 | 0.4 | 0.4 | 0.2 |
| Polyacrylamide *5 | | | | 0.3 | | |
| Acrylates/C10-30 | | | 0.2 | | 0.2 | |
| Alkyl Acrylates | | | | | | |
| Crosspolymer *6 | | | | | | |
| 1,3-ButyleneGlycol | 10 | 10 | 5 | 10 | 8 | 4 |
| Dipropylene Glycol *7 | | | 2 | | | 4 |
| Glycerin | | 3 | 2 | 5 | 5 | 4 |
| Sodium Ascorbyl | 0.5 | | | | 2 | |
| Phosphate *8 | | | | | | |
| Sodium Salicylate | 0.5 | 0.5 | | | | |
| Titanium Dioxide *9 | 0.2 | | 0.1 | | | |
| Polysorbate 20 *10 | 0.3 | 0.3 | 0.2 | 0.3 | 0.3 | 0.2 |
| Perfume | 0.05 | 0.03 | | 0.03 | | 0.03 |
| Methyl Paraben | 0.15 | 0.15 | 0.1 | 0.15 | 0.15 | 0.15 |
| Benzyl Alcohol | 0.25 | 0.25 | 0.25 | | 0.25 | 0.15 |
| EDTA-2Na | 0.1 | 0.1 | 0.1 | 0.1 | 0.1 | 0.1 |
| Saccharomycopsis | | | 30 | | | |
| Fermented Filtrate | | | | | | |
| *11 | | | | | | |

| | |
|---|---|
| Sodium Hydroxide | Adjust pH to 4.5-7.0 |
| Deionized Water | q.s. to 100% |

Definitions of Components
*1 Glucosyl Hesperidin: alpha-Ghesperidin PS-CC, available from Toyo Sugar Refining.
*2 Niacinamide: Niacinamide USP available from DSM
*3 Pyridoxine Hydrochloride: Available from DMS
*4 Xanthan Gum: Keltrol available from Kelco
*5 Polyacrylamide: Sepigel 305 available from SEPPIC Inc
*6 Acrylates/C10-30 Alkyl Acrylates Crosspolymer: Pemulen TR-2 available from B. F. Goodrich
*7 Dipropylene Glycol
*8 Sodium Ascorbyl Phosphate: Stay-C 50 available from DSM
*9 Titanium Dioxide: Kobo GLW75CAP-MP available from Kobo Products Inc.
*10 Polysorbate 20: Tween 20 available from ICI Surfactants
*11 Saccharomycopsis Fermented Filtrate: SKU Pitera available from Kashiwayama

Method of Preparation for Examples 7-12

The compositions above described can be made by any method known to the artisan. The compositions are suitably made as follows:
(1) Any oily component such as perfume, if present, is pre-dissolved into surfactant and a small amount of water.
(2) The water-soluble thickening agent is pre-dispersed into water and/or water-soluble humectant, if present.
(3) All remaining components are dissolved in water and/or water soluble humectant, and the pH is adjusted, as necessary, by a pH adjusting agent. Heat may be added as necessary. When present, relatively oily components such as preservatives can be pre-dissolved in water-soluble humectant.
(4) The products of steps (1) and (3) are mixed until homogeneous.
(5) The products of steps (2) and (4) are mixed until homogeneous, using a high speed mixer as necessary.
(6) The water-insoluble substrate is folded and placed in an aluminium pouch per one unit.
(7) The liquid composition thus obtained at step (5) is poured into the aluminum pouch containing the water-insoluble substrate pouch or preformed gel patch, and hermetically sealed.

### Examples 13-16

Example 13 through 16 are embodiments of gel sheets using a gel sheet composition as specified in Table 3 and a coating composition having the same composition as any of the liquid composition specified in Tables 1 and 2.

TABLE 3

| Ingredient | Ex. 13 | Ex. 14 | Ex. 15 | Ex. 16 |
|---|---|---|---|---|
| Agarose *1 | 0.3 | 0.8 | 1.6 | 1.5 |
| Agar *2 | 0.60 | | | |
| 1:1 Mixture of Xanthan Gum | | 0.5 | 0.8 | 0.75 |
| and Locust Bean Gum *3 | | | | |
| Xanthan *4 | 0.2 | | | |
| Locust Bean Gum *5 | 0.2 | | | |
| Niacinamide *6 | | 5.0 | 8.0 | 10.0 |
| Glucosyl Hesperidin *7 | 5.0 | 0.2 | 3.0 | 0.4 |
| D-Panthenol *8 | 5.0 | | 2.0 | 1.0 |
| Glycerin | 10.0 | 15.0 | 10.0 | 10.0 |
| Disodium EDTA | | 0.10 | 0.10 | 0.10 |
| Butylene Glycol | | 5.0 | | |
| Hexylene Glycol | 3.0 | | 5.0 | 5.0 |
| Ethyl Paraben | 0.20 | 0.15 | 0.15 | 0.15 |

| | |
|---|---|
| Water | Make to total 100% |

Definitions of Components
*1 Agarose: Litex Agarose HSB-LV 2500 available from FMC Bioproducts
*2 Agar: available from Greentech.
*3 1:1 Mixture of Xanthan Gum and Locust Bean Gum: Kelgum available from Kelco
*4 Xanthan: Keltrol T, available from Kelco
*5 Locust Bean Gum: Phytaluronate available from Pentapharm
*6 Niacinamide: Niacinamide USP available from DSM
*7 Glucosyl Hesperidin: alpha-Ghesperidin PS-CC available from Toyo Sugar Refining
*8 D-Panthenol: D-Panthenol USP available from DSM

The pre-formed sheet is made by a continuous line process comprising the steps of:
(1) Dissolve all ingredients into water, mixing and heating to about 80° C. (at least 5° C. higher than the sol-gel transition point) to form a sol phase.
(2) pre-cool the sol phase composition to a temperature of between 0° C. and 5° C. higher than the sol-gel transition point;
(3) form the gel sheet by pouring into a mould or pressing via a roller sheet forming machine with cooling system, cooling the sol phase into a gel phase at temperature no higher than 20° C.;
(4) cutting the gel sheet composition by a die cutter.

The coating composition and preformed gel sheet are placed in the same container and hermetically sealed.

These embodiments represented by the previous examples are useful as skin care products. When applied to the facial skin, they provide many advantages. For example, they can provide improvement in the areas of skin tone, skin lightening, lightening of skin spots, skin sallowness reduction, and fine wrinkle reduction. Significant improvements in the benefits above are observed when the examples are used daily for a period of at least 4 weeks.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care device comprising:
(1) a dermatologically acceptable preformed substrate; and
(2) a flavonoid compound selected from the group consisting of glucosyl hesperidin, glucosyl rutin, glucosyl myricitrin, glucosyl isoquercitrin, glucosyl quercitrin, methyl hesperidin, and mixtures thereof;
wherein the device comprises a liquid medium for delivering the flavonoid compound.

2. The device of claim 1 wherein the liquid medium comprises water.

3. (canceled)

4. The device of claim 1 wherein the flavonoid compound is glucosyl hesperidin.

5. The device of claim 1 wherein the substrate is water-insoluble, and wherein the remainder of the device forms a liquid composition; the liquid composition comprising the liquid medium: wherein the liquid composition comprises by weight of the liquid composition:
(a) from about 0.001% to about 10% of the flavonoid compound
(b) a water soluble thickening agent which provides the liquid composition a viscosity of from about 10 mPas to about 600,000 mPas; and
(c) water; and
wherein the liquid composition is 100% to 2000% by weight of the water-insoluble substrate;

6. The liquid composition of claim 5 further comprising from about 0.01% to about 15% of a vitamin B3 compound by weight of the liquid composition.

7. The liquid composition of claim 6 further comprising from 0.001% to about 15% of a vitamin B6 compound by weight of the liquid composition.

8. The device of claim 1 wherein the substrate is a gel sheet comprising a gelling agent and a hydrophilic solvent, the hydrophilic solvent being the liquid medium; wherein the gel sheet comprises from about 0.001% to about 10% of the flavonoid compound by weight of the gel sheet.

9. The device of claim 8 wherein the device further comprises a coating composition, the coating composition comprising from about 0.001% to about 10% of the flavonoid compound by weight of the coating composition.

10. The device of claim 9 wherein the gel sheet and coating composition each further comprise from about 0.01% to about 15% of a vitamin B3 compound.

11. The device of claim 10 wherein the gel sheet and coating composition each further comprise from 0.001% to about 15% of a vitamin B6 compound.

12. A method of lightening the skin comprising the step of applying to the skin a device comprising:
(1) a dermatologically acceptable performed substrate; and
(2) a flavonoid compound selected from the group consisting of glucosyl hesperidin, glucosyl rutin, glucosyl myricitrin, glucosyl isoquercitrin, glucosyl quercitrin, methyl hesperidin, and mixtures thereof;
(3) a liquid medium for delivering the flavonoid compound.

13. A method of providing anti-aging benefit to the skin comprising the step of applying to the skin a device comprising:
(1) a dermatologically acceptable preformed substrate; and
(2) a flavonoid compound selected from the group consisting of glucosyl hesperidin, glucosyl rutin, glucosyl myricitrin, glucosyl isoquercitrin, glucosyl quercitrin, methyl hesperidin, and mixtures thereof;
(3) a liquid medium for delivering the flavonoid compound.

14. The method of Claim 12 wherein the device is applied to the skin for at least 5 minutes.

15. The method of Claim 13, wherein the device is applied to the skin for at least 5 minutes.
